SEQUENCE LISTING (10 sequences)

SEQ ID NO: 1 - protein, 11 aa, synthetic construct
NSPRRARSVAS

SEQ ID NO: 2 - protein, 8 aa, synthetic construct
NSPASVAS

SEQ ID NO: 3 - DNA, 3810 bp, synthetic construct
  source 1..3810: mol_type = other DNA
atgttcgtattccttgtcctcctccccctcgtcagctcccagtgtgtcaatctgacaactcgaactcagctcccacccgcatacaccaatagttttacccgaggagtctattatcccgacaaagttttcaggtctagtgtgttgcatagcacgcaggacctgttcttgccgtttttttcaaatgtgacatggttccatgccatccatgtctctggtacgaacgggaccaaaaggtttgataaccccgttcttccatttaacgacggggtttatttcgcctctactgaaaagtctaatataatacgaggttggatttttgggacaacactggactctaaaacgcaatctctcctcatagtaaataatgcgacgaatgtcgtaatcaaagtatgtgagtttcagttttgcaacgacccttttcttggtgtatactatcacaagaacaacaagtcttggatggaaagtgaatttagagtctacagctccgcaaacaactgtacctttgagtacgtcagtcagcccttccttatggacctcgaaggcaaacaggggaacttcaagaatctgcgcgagttcgtgttcaagaatatcgacggctatttcaagatatattcaaaacatacaccaatcaatttggtacgcgatttgccgcagggcttctctgctcttgagcctctcgtagatttgcctatagggatcaatattactcgctttcaaactctccttgcccttcatcggtcctacctgacaccaggcgacagctccagtgggtggacagctggtgcagcggcttactacgtcggctacctccaacccaggacttttttgctgaaatataatgagaatggtacgattactgacgccgtcgattgtgccctcgacccattgagcgaaactaaatgcaccctgaaaagctttacggttgagaaagggatatatcagacatctaattttcgcgttcaacccacggaatccattgtacgatttcccaacattaccaatttgtgtccttttggtgaggtatttaatgcaaccagattcgcgagtgtgtatgcctggaataggaaaagaatttctaactgtgtagcagactacagcgttctgtataatagtgcgtcctttagtacgttcaagtgctacggagtaagcccgacaaagttgaacgatttgtgtttcactaacgtctatgccgatagcttcgtcatcaggggagacgaagtaaggcaaattgcaccaggccagaccggaaaaatagccgactacaactataagttgccggacgatttcaccgggtgtgtcatcgcgtggaatagtaataacctggactccaaggtgggcggcaactacaactacctgtaccgattgttccgcaagtctaacctcaagccctttgaacgagacatatccaccgagatataccaagcgggaagcactccctgtaatggcgttgaaggtttcaactgctattttcctctccagtcctatggcttccaacctaccaatggagtaggctatcaaccgtatcgagtcgtggtgctgagtttcgagctcctgcacgctccagcgaccgtttgtggtcctaagaaaagtacaaacctcgtaaaaaataagtgcgtaaactttaactttaacggtctcacaggaacgggagttcttacagagtccaataagaagttcctgccctttcagcagtttgggcgcgacatcgcggacaccacagatgcagtcagggacccgcagactcttgagatcctggacatcaccccatgctcttttggcggtgtgagtgtcattacacccgggaccaacaccagcaatcaggttgctgtgctctatcaagacgtcaactgtacggaagtcccagttgcaatacacgcggaccagttgacgccgacgtggcgggtttattctaccggctcaaacgtctttcaaactagagccggttgtttgatcggggcggagcatgtgaacaattcatacgaatgtgatattcccataggagccggaatatgtgcttcttaccagacccaaaccaactctccggcatccgtagccagtcaaagcataattgcgtacaccatgagcctcggtgcagaaaactcagttgcgtactcaaataactccattgccatcccaacaaattttaccatatcagtgaccactgaaatccttccagtgagcatgactaaaacaagtgtagattgcacaatgtatatttgcggagactcaactgagtgctctaacctcctgttgcagtacggttcattctgtacccagctcaatcgcgcacttacgggaatagcagtagaacaagataagaatactcaggaagtcttcgctcaagtaaagcaaatctacaagacgccccccattaaggactttgggggttttaattttagccaaatactcccggaccccagtaaaccctctaagaggtcatttatagaagacctgttgtttaataaagttactctggccgatgctggctttattaaacaatacggtgattgtttgggcgacatcgcggcgcgggatctgatatgcgcccaaaaatttaacggtctcactgtgttgccgcctttgctcacagacgagatgatagctcagtacacatcagcgttgttggcgggtaccataacgtctggatggacattcggagcaggggcggccttgcaaataccattcgcaatgcaaatggcatacaggttcaacgggataggtgttacacagaatgtactttacgagaaccagaaattgattgcaaaccagtttaactctgcgattggcaaaattcaggatagcctcagctccaccgcgtctgcactgggaaaactccaagatgttgtgaaccaaaatgcccaagccctcaatacacttgtgaagcaactctccagcaatttcggtgcaataagtagtgtgctgaacgacatactttctcggttggacaaagtagaggcggaggtgcaaatcgacagactcataacaggtcgactgcaatcattgcaaacgtatgttactcagcaattgatacgggcggcggagattcgagcgtctgccaacctcgccgcgactaaaatgtctgagtgtgtgctcggtcagtcaaaacgcgtggacttctgtggaaagggataccatctcatgtccttccctcagtctgcccctcacggagtagtctttcttcatgtgacctatgttccagcacaggagaaaaattttaccacagcgcccgcgatatgccacgatggcaaagctcattttccgcgagagggagtatttgtctctaacggcacccattggtttgtaacccagcgcaatttttacgaacctcagataatcaccactgacaacacctttgttagtggtaattgtgatgtagtgatcgggatagtgaacaatacagtgtacgaccccctgcaacctgagctggactcctttaaagaagaactcgacaagtacttcaaaaaccacacttccccggatgtcgatttgggtgatatatctggcatcaatgcgagtgtagtcaacatacagaaggaaatcgacaggctcaatgaagtagcaaaaaacctgaatgaatccctcatagacctccaagagctgggcaaatatgagcagtacattaaatggccctggtacatttggctgggcttcatcgctggcctgatagcgatagtgatggttactattatgctctgctgtatgacgagttgttgcagctgcctcaaagggtgttgttcttgcggctcctgttgtaagttcgacgaggatgattcagagcccgttcttaagggtgtaaaactccattacact

SEQ ID NO: 4 - protein, 1270 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 669 bp, synthetic construct
  source 1..669: mol_type = other DNA
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 6 - DNA, 228 bp, synthetic construct
  source 1..228: mol_type = other DNA
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 7 - DNA, 1257 bp, synthetic construct
  source 1..1257: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaaaatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcacaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 8 - protein, 222 aa, synthetic construct
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 9 - protein, 75 aa, synthetic construct
MYSFVSEETGTLIVNSVLLFLAFVVFLLVTLAILTALRLCAYCCNIVNVSLVKPSFYVYSRVKNLNSSRVPDLLV

SEQ ID NO: 10 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

(19) United States
(12) Patent Application Publication
(10) Pub. No.: US 2025/0000967 A1
(43) Pub. Date: Jan. 2, 2025

(54) EXOSOME-BASED ANTIVIRAL VACCINE AND MANUFACTURING METHOD THEREOF

(71) Applicant: CK-EXOGENE CO., LTD., Jeollabuk-do (KR)

(72) Inventors: Jae Young KIM, Seoul (KR); Yong Joon CHWAE, Gyeonggi-do (KR); Tamanna MUSTAJAB, Gyeonggi-do (KR)

(21) Appl. No.: 18/708,756

(22) PCT Filed: Nov. 10, 2022
(86) PCT No.: PCT/KR2022/017672

§ 371 Date: Jul. 9, 2024

(30) Foreign Application Priority Data
Nov. 10, 2021 (KR) 10-2021-0154283
Dec. 29, 2021 (KR) 10-2021-0191702

(51) Int. Cl. A61K 39/215; A61P 31/14; C12N 7/00

(52) U.S. Cl. CPC A61K 39/215; A61P 31/14; C12N 7/00; C12N 2770/20023; C12N 2770/20034

(57) ABSTRACT

The present invention relates to an exosome platform-based antiviral vaccine. with the ability to induce a strong immune response to viruses and induce a stable and long-term immune response even to viruses with frequent mutations, the exosome platform-based antiviral vaccine can be utilized effectively for use as an antiviral vaccine.

### TECHNICAL FIELD

The present invention relates to an exosome-based anti-viral vaccine.

### BACKGROUND ART

COVID-19, which is currently a global problem, is defined as a respiratory syndrome caused by the SARS-COV-2 virus infection. The pathogen, SARS-COV-2, is an RNA virus belonging to the Coronaviridae family. The primary transmission routes are currently known to be airborne (droplets) and contact. Severe illness or death is more common in older adults, immunocompromised patients, and patients with underlying conditions, and there is currently no suitable treatment other than conservative treatment such as fluid supplementation and antipyretic drugs.

To combat this viral infection, vaccines have been developed and administered. However, there is a problem that the immunity wanes rapidly over time after vaccination, necessitating continuous additional vaccinations (booster shots) seasonally or at regular intervals. In addition, breakthrough infections occur even after vaccination due to the inability of the existing immune system due to the continuous occurrence of variants. Therefore, the development of new vaccine materials that can induce strong immunity to respond to these variants is required.

On the other hand, apoptotic exosomes (extracellular vesicles) express CD63, similar to exosomes secreted from healthy cells, and have specific markers, Spingoshine-1-phosphate Receptor 1 and Spingoshine-1-phosphate Receptor 3. Apoptotic exosomes are reported to be involved in cell-to-cell interactions and immune responses, but the details of their biosynthesis are not fully understood. Furthermore, research is ongoing to develop antiviral vaccines using exosomes, but there have been no successful cases to date.

Therefore, the present invention has been completed through research on the development of an antiviral vaccine using exosomes.

### DISCLOSURE

### Technical Problem

One aspect of the present invention is to provide exosomes comprising a viral structural protein.

Another aspect of the present invention is to provide a viral vaccine composition comprising the exosomes.

Yet another aspect of the present invention is to provide a cell line for producing the exosomes.

### Technical Solution

The invention provides exosomes comprising a viral structural protein.

The exosomes of the present invention can be used with a pharmaceutically acceptable carrier. Pharmaceutically acceptable carriers that can be used with the exosomes of the present invention are those conventionally used in the preparation of pharmaceuticals, and include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, and mineral oil. In addition to the above ingredients, the exosomes of the present invention may further include lubricants, humectants, sweeteners, flavorings, emulsifiers, suspending agents, preservatives, and the like. Suitable pharmaceutically acceptable carriers and formulations are described in detail in Remington: the science and practice of pharmacy 22nd edition (2013).

The exosomes of the present invention can include various additives and/or adjuvants that are necessary and appropriate for formulation, and can be prepared by further including known compounds within a range that does not impair their effect such as nonionic surfactants, silicone polymers, pigments, fragrances, preservatives, antimicrobials, antioxidants, organic solvents, ionic or nonionic thickeners, plasticizers, antioxidants, free radical scavengers, opacifiers, stabilizers, emollients, silicones, α-hydroxy acids, vesicants, moisturizers, vitamins, insect repellents, fragrances, preservatives, surfactants, anti-inflammatory agents, substance P antagonists, fillers, polymers, propellants, alkalizing or acidifying agents, or coloring agents.

The appropriate dosage of the exosomes of the present invention can vary widely depending on factors such as the method of formulation, the route of administration, the age, weight, sex, medical condition, diet, time of administration, route of administration, rate of excretion, and response sensitivity of the patient, and may be, for example, 0.001 to 1000 mg/kg for an adult.

The exosomes of the present invention can be administered parenterally, intramuscularly, subcutaneously, and nasally, and can be administered by methods such as subcutaneous injection, intravenous injection, intramuscular injection, or nasal (topical or inhaled) administration, but are not limited thereto.

The formulation of the invention for non-parenteral administration can be prepared, for example, by mixing the exosomes of the present invention with a stabilizing agent or buffer in water to make a solution or suspension, and formulating it into an ampule or vial for unit dosage administration. In addition, preservatives, stabilizers, humectants or emulsification promoters, salts and buffers for osmotic pressure adjustment, and other therapeutically useful substances may be further added, and the composition may be formulated by conventional methods.

These formulations can be presented in unit-dose (single-use) or multi-dose (multiple-use) containers, spray dispensers, inhalation containers, such as sealed ampules and vials, and can be stored under freeze-dried conditions requiring only the addition of a sterile liquid vehicle, such as water for injection, immediately before use. Instantaneous use formulations and suspensions can be prepared from sterile powders, granules and tablets.

According to one embodiment of the present invention, the exosomes can be general exosomes and apoptotic exosomes.

According to one embodiment of the present invention, the apoptosis can be induced by cleavage of a Gasdermin family protein.

According to one embodiment of the present invention, the cleavage can be induced by staurosporine.

According to one embodiment of the present invention, the culture medium for obtaining the exosomes may comprise one or more substances selected from the group consisting of tumor necrosis factor alpha (TNF-α), Cycloheximide, Anisomycin, Aurintricarboxylic acid, Diphtheria toxin, Edeine, Fusidic acid, Pactamycin, Puromycin, Ricin, Sodium fluoride, Sparsomycin, Tetracycline, Trichoderma and staurosporine.

According to one embodiment of the present invention, the Gasdermin family protein can be one or more proteins selected from the group consisting of GSDMA, GSDMB, GSDMC, GSDMD, DFNA5 (GSDME), and DFNB59.

GSDMD (GasderminD) is cleaved by caspase activated by the inflammasome, separating the N-terminal domain from the C-terminal inhibitory domain, and the separated N-terminal domain oligomerizes on the cell membrane to form a pore with a diameter of 10 to 16 nm. Through this pore, the inflammatory cytokines IL-1 and IL-18 are secreted, and eventually pyroptosis occurs with cell membrane rupture and release of intracellular components to the extracellular space.

In addition, GSDME (GasderminE), which is expressed by the DFNA5 gene, is cleaved by caspase 3 activated during apoptosis to induce secondary necrosis or pyroptosis, which perforates the cell membrane and releases intracellular components to the extracellular space.

In the present invention, the term “Pyroptosis” refers to a type of cell death characterized by pore formation in the plasma membrane, cell swelling, and plasma membrane disruption, similar to necrosis but not apoptosis.

In the present invention, the term “inflammasome” refers to a substance that is a molecular mechanism that induces the maturation of inflammatory cytokines such as IL-1, which is related to the innate immune defense system of cells against infection or stress.

In the present invention, the term “cytokine” refers to a protein secreted by immune cells. After being secreted from cells, cytokines can influence other cells or the cells that secreted them. For example, cytokines can induce the proliferation of macrophages or promote the differentiation of the secreting cells themselves.

The SARS-COV2 structural proteins M, E, N and S of the present invention can be cloned into recombinant expression vectors containing the nucleotide sequences encoding them and expressed in host cells.

The term “recombinant expression vector” used in the present invention means a recombinant DNA molecule that includes a desired coding sequence and appropriate nucleic acid sequences essential for expressing the coding sequence operably linked to a specific host organism. Promoters, enhancers, terminators, and polyadenylation signals available in eukaryotic cells are known.

In the context of this invention, the term “functionally ligated” refers to the functional joining of a gene expression regulatory sequence to a different nucleotide sequence. The gene expression regulatory sequence can be one or more selected from a group consisting of a replication origin, a promoter, and a terminator sequence. The terminator sequence can be a polyadenylation sequence (pA), and the replication origin can be a fl replication origin, an SV40 replication origin, a pMB1 replication origin, an adenovirus replication origin, an AAV replication origin, or a BBV replication origin, but is not limited thereto.

As used herein, the term “promoter” refers to a region upstream of a structural gene, and refers to a DNA molecule to which RNA polymerase binds to initiate transcription.

In one embodiment of the present invention, the promoter can be a polynucleotide fragment of about 100 bp to about 2,500 bp in length, which is one of the transcription regulatory sequences that control the transcription initiation of a specific gene. For example, the promoter can be selected from a group consisting of a CMV promoter (cytomegalovirus promoter (e.g., human or mouse CMV immediate-early promoter)), a U6 promoter, an EF1-alpha (elongation factor 1-a) promoter, an EF1-alpha short (EFS) promoter, an SV40 promoter, an adenovirus promoter (major late promoter), a pL λ promoter, a trp promoter, a lac promoter, a tac promoter, a T7 promoter, a vaccinia virus 7.5K promoter, an HSV tk promoter, an SV40E1 promoter, a respiratory syncytial virus (RSV) promoter, a metallothionin promoter, a β-actin promoter, a ubiquitin C promoter, a human IL-2 (human interleukin-2) gene promoter, a human lymphotoxin gene promoter, and a human GM-CSF (human granulocyte-macrophage colony stimulating factor) gene promoter, but is not limited thereto.

According to one embodiment of the present invention, the recombinant expression vector can be selected from a group consisting of plasmid vectors, cosmid vectors, and bacteriophage vectors, viral vectors such as adenovirus vectors, retrovirus vectors, and adeno-associated virus vectors. Vectors that can be used as recombinant expression vectors can be prepared based on plasmids (e.g., pcDNA series, pSC101, pGVI106, pACYC177, ColEI, pKT230, pME290, pBR322, pUC8/9, pUC6, pBD9, pHC79, plJ61, pLAFRI, pHV14, pGEX series, pET series, pUC19, etc.), phages (e.g., λgt4λB, λ-Charon, λΔz1, M13, etc), or viral vectors (e.g., adeno-associated virus (AAV) vectors, etc.) used in the industry, but is not limited thereto.

The recombinant expression vector of the present invention may further comprise one or more selectable markers. The markers are nucleic acid sequences that typically have a characteristic that can be selected by chemical means, and include all genes that can distinguish transformed cells from non-transformed cells. For example, they can be herbicide resistance genes such as glyphosate, glufosinate ammonium, or phosphinothricin, or antibiotic resistance genes such as ampicillin, kanamycin, G418, bleomycin, hygromycin, chloramphenicol, puromycin, blastidin, and zeocin, but are not limited thereto.

The recombinant expression vector of the present invention can be constructed using well-known gene recombination techniques in the art, and site-specific DNA cleavage and ligation can be performed using enzymes generally known in the art.

According to one embodiment of the present invention, the exosomes can be derived from cells selected from the group consisting of stem cells, immune cells, somatic cells, fetal cells, and tumor cells.

According to one embodiment of the present invention, the exosomes can be derived from a human cell line or an animal cell line.

The stem cells can be mesoderm stem cells, pluripotent stem cells, multipotent stem cells, or unipotent stem cells, but are not limited thereto.

The pluripotent stem cells can be embryonic stem cells (ES Cell), undifferentiated germline cells (EG Cell), reprogrammed stem cells (IPS Cell), or induced pluripotent stem cells (iPSC), but are not limited thereto.

The multipotent stem cells can be adult stem cells such as mesenchymal stem cells (derived from adipose, bone marrow, umbilical cord, or umbilical cord blood), hematopoietic stem cells (derived from bone marrow or peripheral blood), neural stem cells, and germline stem cells, but are not limited thereto.

The mesenchymal stem cells can be human embryonic stem cell-derived mesenchymal stromal cells (hES-MSC), bone marrow mesenchymal stem cells (BM-MSC), umbilical cord mesenchymal stem cells (UC-MSC), or Adipose-Derived Stem Cell-Conditioned Medium (ADSC), but are not limited thereto.

The stem cells can be autologous or allogeneic stem cells.

The immune cells can be selected from the group consisting of dendritic cells, natural killer cells, T cells, B cells, regulatory T cells (Treg cells), natural killer T cells, innate lymphoid cells, macrophages, granulocytes, chimeric antigen receptor-T cells (CAR-T cells), lymphokine-activated killer cells (LAK), and cytokine-induced killer cells (CIK), but are not limited thereto.

The somatic cells can be selected from the group consisting of fibroblasts, chondrocytes, synovial cells, keratinocytes, adipocytes, osteoblasts, osteoclasts, and peripheral blood mononuclear cells (PBMCs), but are not limited thereto.

The cell lines can be selected from the group consisting of CHO cells, NS0 cells, Sp2/0 cells, BHK cells, C127 cells, HEK293 cells, HEK293T cells, HEK-293 STF cells, 293T/17 cells, 293T/17 SF cells, or HEK-2932sus cells, HT-1080 cells, PERC6 cells, NuLi-1 cells, ARPE-19 cells, VK2/E6E7 cells, Ectl/E6E7 cells, RWPE-2 cells, WPE-stem cells, End1/E6E7 cells, WPMY-1 cells, NL20 cells, NL20-TA cells, WT 9-7 cells, WPE1-NB26 cells, WPE-int cells, RWPE2-W99 cells, HaCaT cells, hTERT-immortalized human fibroblast cells, BJ-5ta cells, and BEAS-2B cells, but are not limited thereto.

The tumor cells can be cells derived from ovarian cancer, breast cancer, liver cancer, brain cancer, colon cancer, prostate cancer, cervical cancer, lung cancer, stomach cancer, skin cancer, pancreatic cancer, oral cancer, rectal cancer, laryngeal cancer, thyroid cancer, parathyroid cancer, colon cancer, bladder cancer, peritoneal cancer, adrenal cancer, tongue cancer, small intestine cancer, esophageal cancer, kidney cancer, renal cancer, heart cancer, duodenal cancer, ureteral cancer, urethral cancer, pharyngeal cancer, vaginal cancer, tonsil cancer, anal cancer, pleural cancer, thymic cancer, or nasopharyngeal cancer, but are not limited thereto. Specifically, The tumor cells can be selected from the group consisting of human ovarian cancer cell lines (SKOV3, OVCAR3), human breast cancer cell lines (MCF-7, T47D, BT-474), human hepatocellular carcinoma cell lines (Hep3B, HepG2), human glioblastoma cell lines (U87MG, U251), human colon cancer cell lines (SW480, HT-29, HCT116, Caco-2), human lung cancer cell lines (A549, NCIH358, NCI-H460), human prostate cancer cell lines (22RV1), human cervical cancer cell lines (HeLa), human melanoma cell lines (A375), human embryonic kidney cell lines (HEK 293T), and human gastric cancer cell lines (NCI-N87), but are not limited thereto.

According to one embodiment of the present invention, the structural protein can be one or more proteins selected from the group consisting of membrane, envelope, nucleocapsid, and spike.

The amino acid sequences and nucleic acid sequences that encode the structural proteins of the present invention, for example, the membrane protein, envelope protein, nucleocapsid protein, and spike protein of the virus, can be easily selected by those skilled in the art by searching publicly known databases, such as GenBank (www.ncbi.nlm.nih.gov/genbank/).

Another aspect of the present invention provides a viral vaccine composition comprising the exosomes.

The virus can be an influenza virus. The influenza virus can be a virus belonging to the Orthomyxoviridae family, which is an RNA virus, and specifically can be a virus of the genus Influenza A, Influenza B, and/or Influenza C, and can include a virus having a glycoprotein called hemagglutinin (HA) and neuraminidase (NA) on the surface of the viral particle, such as H1N1 subtype, H3N2 subtype, H5N1 subtype, and H7N7 subtype, but is not limited thereto.

The virus can be an RNA virus belonging to the Coronavirinae subfamily. Specifically, it can include HCOV-229E, HCoV-OC43, SARS-COV, HCoV-NL63, MERS-CoV, and SARS-COV-2, but is not limited thereto.

According to one embodiment of the present invention, the virus can be a coronavirus.

According to one embodiment of the present invention, the coronavirus can be SARS-COV-2.

The exosomes of the present invention can effectively overcome the vaccine breakthrough infection problem caused by SARS-COV-2 variants, as they can include not only the frequently mutated spike protein but also the more conserved and stable membrane, envelope, and/or nucleocapsid proteins.

Another aspect of the present invention provides a cell line for producing the exosomes.

In order to prepare a transformed cell line into which a recombinant expression vector according to one embodiment of the present invention has been introduced, methods known in the art for introducing a nucleic acid molecule into an organism, cell, tissue, or organ can be used, and an appropriate standard technique can be selected according to the host cell as known in the art. Such methods may include electroporation, calcium phosphate (CaPO₄) precipitation, calcium chloride (CaCl₂) precipitation, microinjection, polyethylene glycol (PEG) method, DEAE-dextran method, cationic liposome method, and lithium acetate-DMSO method, but are not limited thereto.

Exosomes produced from cell lines into which the recombinant expression vector of the present invention has been introduced can be obtained by conventional methods by culturing the cell lines by conventional methods.

The cells that can be used as transformed cell lines can be Escherichia coli, but are not limited thereto.

### Advantageous Effects

According to the apoptotic exosome platform-based antiviral vaccine, it can be effectively used for antiviral vaccines as it can induce a strong immune response to viruses and induce a stable and long-term immune response even to frequently mutated viruses.

### BRIEF DESCRIPTION OF DRAWINGS

FIG. 1 is a diagram illustrating an overview of a SARS-COV-2 structural protein-expressing apoptotic exosome-based COVID19 vaccine.

FIG. 2 is a diagram showing the DNA sequence of the SARS-COV-2 spike protein 3R mutant.

FIG. 3 is a diagram showing the amino acid sequence encoded by the DNA sequence of the SARS-COV-2 spike protein 3R mutant.

FIG. 4 is a diagram showing the structure of the SARS-COV-2 spike protein 3R mutant according to one embodiment of the present invention.

FIG. 5 is a graph showing the structure of a SARS-COV-2 structural protein cDNA expression construct according to one embodiment of the present invention.

FIG. 6 is a schematic diagram showing a separation protocol for apoptotic exosomes according to one embodiment of the present invention.

FIG. 7 is a photograph showing the results of western blot analysis of SARS-CoV-2 structural proteins and exosome markers in apoptotic exosomes according to one embodiment of the present invention.

FIG. 8 is a graph showing the results of antibody formation ability analysis of apoptotic exosomes according to one embodiment of the present invention.

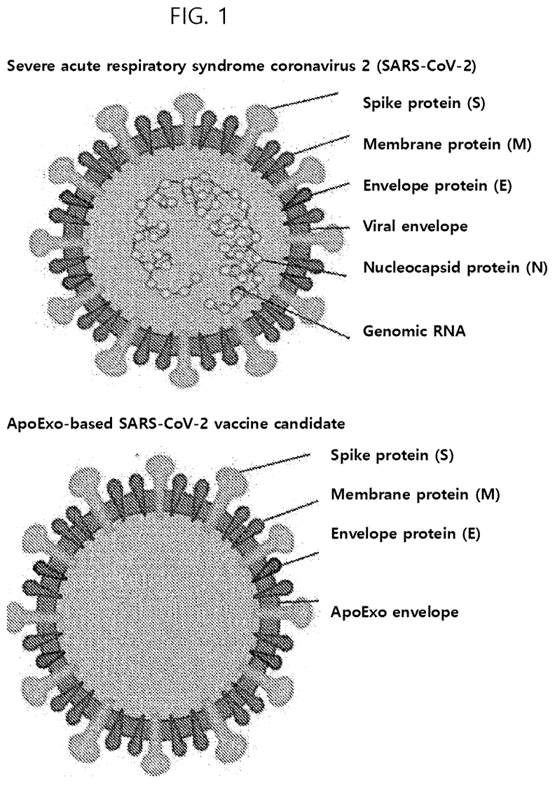

### MODE FOR INVENTION

The present invention will be described in more detail with reference to one or more embodiments. However, these embodiments are intended to illustrate the present invention and the scope of the present invention is not limited to these embodiments.

### Example 1: Preparation of Lentivirus-Like Particles Containing SARCs-CoV-2-cDNA

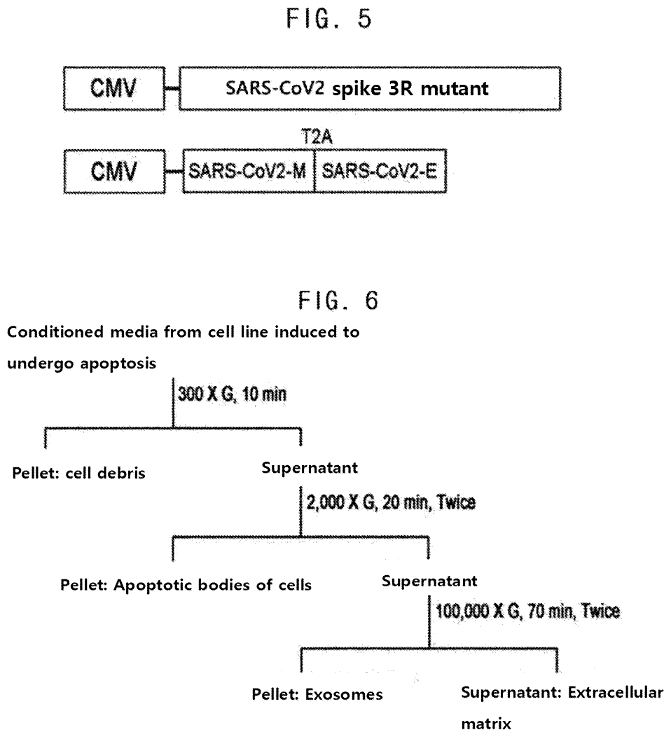

In order to prepare lentiviral-like particles containing SARS-COV-2-cDNA, SARS-COV2-M-T2A-E (Membrane-T2A-Envelope), SARS-COV2-N, and SARS-COV2-S (Spike) cDNA were generated under the control of the CMV promoter (FIG. 5). Subsequently, the prepared SARS-COV2-M-T2A-E and SARS-COV2-N cDNA were cloned into pCDH-CMV-MCS-EF1-Blastidin and pCDH-CMV-MCS-EF1-Puromycin lentiviral vectors, respectively.

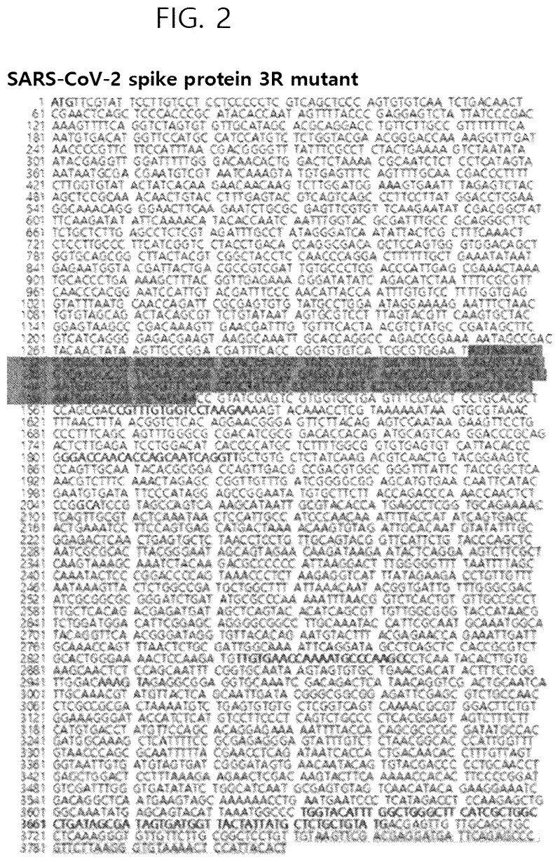

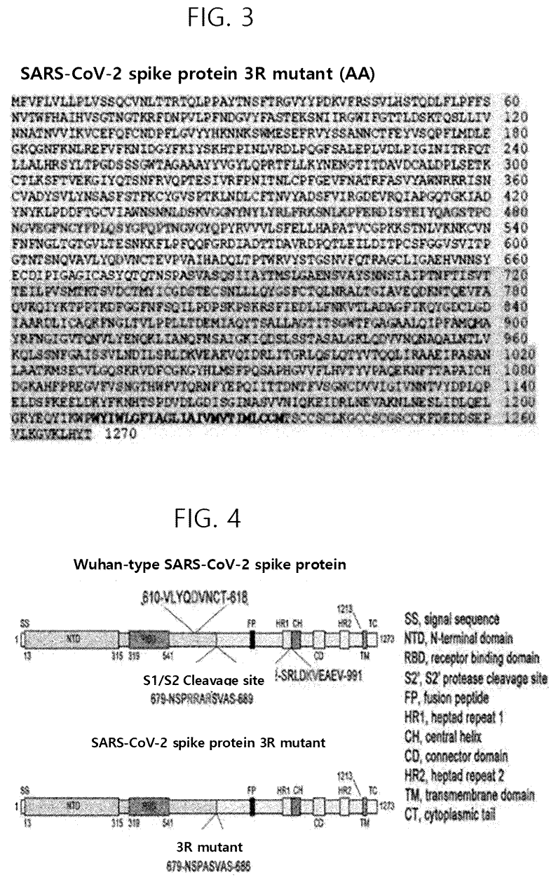

In addition, for SARS-COV2, a SARS-COV2-S 3R mutant (SEQ ID NO 4, FIG. 3) was preparated by introducing a mutation in the S1/S2 cleavage site, resulting in the deletion of three R residues in the amino acid sequence NSPRRARSVAS (SEQ ID NO 1) at positions 679 to 689. The corresponding DNA sequence (SEQ ID NO 3, FIG. 2) encoding this mutant was cloned into the pCDH-CMV-MCS-EF1-Zeocin vector.

TABLE 1

| Name | Amino Acid Sequence (N−>S) | SEQ ID |
|---|---|---|
| SARS-CoV2-S(679-689) | 679-NSPRRARSVAS-689 | SEQ ID 1 |
| SARS-CoV2-S 3R | 679-NSPASVAS-686 | SEQ ID 2 |
| mutant(679-686) | | |

The amino acid sequences of SARS-COV2-M, SARS-COV2-E, and SARS-CoV2-N, and the corresponding DNA sequences encoding them, are as follows.

TABLE 2

| Name | DNA Sequence (5′->3′) | SEQ ID |
|---|---|---|
| SARS-CoV | ATGGCAGATTCCAACGGTACTATTA | SEQ ID 5 |
| 2-M | CCGTTGAAGAGCTTAAAAAGCTCCT | |
| | TGAACAATGGAACCTAGTAATAGGT | |
| | TTCCTATTCCTTACATGGATTTGTC | |
| | TTCTACAATTTGCCTATGCCAACAG | |
| | GAATAGGTTTTTGTATATAATTAAG | |
| | TTAATTTTCCTCTGGCTGTTATGGC | |
| | CAGTAACTTTAGCTTGTTTTGTGCT | |
| | TGCTGCTGTTTACAGAATAAATTGG | |
| | ATCACCGGTGGAATTGCTATCGCAA | |
| | TGGCTTGTCTTGTAGGCTTGATGTG | |
| | GCTCAGCTACTTCATTGCTTCTTTC | |
| | AGACTGTTTGCGCGTACGCGTTCCA | |
| | TGTGGTCATTCAATCCAGAAACTAA | |
| | CATTCTTCTCAACGTGCCACTCCAT | |
| | GGCACTATTCTGACCAGACCGCTTC | |
| | TAGAAAGTGAACTCGTAATCGGAGC | |
| | TGTGATGCTTCGTGGACATCTTCGT | |
| | ATTGCTGGACACCATCTAGGACGCT | |
| | GTGACATCAAGGACCTGCCTAAAGA | |
| | AATCACTGTTGCTACATCACGAACG | |
| | CTTTCTTATTACAAATTGGGAGCTT | |
| | CGCAGCGTGTAGCAGGTGACTCAGG | |
| | TTTTGCTGCATACAGTCGCTACAGG | |
| | ATTGGCAACTATAAATTAAACACAG | |
| | ACCATTCCAGTAGCAGTGACAATAT | |
| | TGCTTTGCTTGTACAGTAA | |
| SARS-CoV | ATGTACTCATTCGTTTCGGAAGAGA | SEQ ID 6 |
| 2-E | CAGGTACGTTAATAGTTAATAGCGT | |
| | ACTTCTTTTTCTTGCTTTCGTGGTA | |
| | TTCTTGCTAGTTACACTAGCCATCC | |
| | TTACTGCGCTTCGATTGTGTGCGTA | |
| | CTGCTGCAATATTGTTAACGTGAGT | |
| | CTTGTAAAACCTTCTTTTTACGTTT | |
| | ACTCTCGTGTTAAAAATCTGAATTC | |
| | TTCTAGAGTTCCTGATCTTCTGGTC | |
| | TAA | |
| SARS-CoV | ATGTCTGATAATGGACCCCAAAATC | SEQ ID 7 |
| 2-N | AGCGAAATGCACCCCCCATTACGTT | |
| | TGGTGGACCCTCAGATTCAACTGGC | |
| | AGTAACCAGAATGGAGAACGCAGTG | |
| | GGGCGCGATCAAAACAACGTCGGCC | |
| | CCAAGGTTTACCCAATAATACTGCG | |
| | TCTTGGTTCACCGCTCTCACTCAAC | |
| | ATGGCAAGGAAGACCTTAAATTCCC | |
| | TCGAGGACAAGGCGTTCCAATTAAC | |
| | ACCAATAGCAGTCCAGATGACCAAA | |
| | TTGGCTACTACCGAAGAGCTACCAG | |
| | ACGAATTCGTGGTGGTGACGGTAAA | |
| | ATGAAAGATCTCAGTCCAAGATG | |

TABLE 3

| Name | Amino Acid Sequence (N−>S) | SEQ ID |
|---|---|---|
| SARS-CoV2-M(AA) | MADSNGTITVEELKKLLEQWNLVIGFLFLTWICLLQFAYANRNRFLY | SEQ ID 8 |
| | IIKLIFLWLLWPVTLACFVLAAVYRINWITGGIAIAMACLVGLMWLS | |
| | YFIASFRLFARTRSMWSFNPETNILLNVPLHGTILTRPLLESELVIG | |
| | AVILRGHLRIAGHHLGRCDIKDLPKEITVATSRTLSYYKLGASQRVA | |
| | GDSGFAAYSRYRIGNYKLNTDHSSSSDNIALLVQ | |
| SARS-CoV2-E(AA) | MYSFVSEETGTLIVNSVLLFLAFVVFLLVTLAILTALRLCAYCCNIV | SEQ ID 9 |
| | NVSLVKPSFYVYSRVKNLNSSRVPDLLV | |
| SARS-CoV2-N(AA) | MSDNGPQNQRNAPRITFGGPSDSTGSNQNGERSGARSKQRRPQGLPN | SEQ ID 10 |
| | NTASWFTALTQHGKEDLKFPRGQGVPINTNSSPDDQIGYRRATRRIR | |
| | GGDGKMKDLSPRWYFYYLGTGPEAGLPYGANKDGIIWVATEGALNTP | |
| | KDHIGTRNPANNAAIVLQLPQGTTLPKGFYAEGSRGGSQASSRSSSR | |
| | SRNSSRNSTPGSSRGTSPARMAGNGGDAALALLLLDRLNQLESKMSG | |
| | KGQQQQGQTVTKKSAAEASKKPRQKRTATKAYNVTQAFGRRGPEQTQ | |
| | GNFGDQELIRQGTDYKHWPQIAQFAPSASAFFGMSRIGMEVTPSGTW | |
| | LTYTGAIKLDDKDPNFKDQVILLNKHIDAYKTFPPTEPKKDKKKKAD | |
| | ETQALPQRQKKQQTVTLLPAADLDDFSKQLQQSMSSADSTQA | |

Meanwhile, HEK293 cells were prepared by culturing in DMEM containing 10% FBS, 2 mM L-glutamine, 100U/mL penicillin, and 100U/mL streptomycin. The vectors containing The SARS-COV2-M-T2A-E cDNA, SARS-COV2-N cDNA, and SARS-COV2-S cDNA were transfected into the prepared 293T cells along with pGagpol and pVSVg. After 48 and 72 hours, pseudovirus particles containing SARS-COV-2 structural protein cDNA were obtained, respectively.

### Example 2: Establishment of Cell Lines Modified to Induce Apoptosis and Exosome Secretion

To prepare a cell lines modified to induce apoptosis and exosome secretion, 293T (human embryonic kidney, System Biosciences) cells were cultured in minimal essential medium (MEM) supplemented with 10% heat-inactivated FBS, 2 mM L-glutamine, 100U/mL penicillin, and 100 ug/mL streptomycin. cDNA of Gasdermin family genes GSDMA (Sino biological, Beijing, China), GSDMB (Sino biological, Beijing, China), GSDMC (Sino biological, Beijing, China), GSDMD (Sinobiotic, Beijing, China), DFNA5 (GSDME, Sinobiotic, Beijing, China), or DFNB59 cDNA (Sino biological, Beijing, China) was subcloned into the pCDH-EFI-MCS-T2A-puro Lentiviral vector (System Biosciences). All lentiviral vectors were infected into 293T cells using Lipofectamine 2000 transfection reagents (Invitrogen). After 2 days, pseudovirus particles were collected and infected into 293T cells. After selection with puromycin for 2 weeks, cell lines that secrete exosomes through apoptosis were established.

### Example 3: Preparation of SARS-COV-2 Gene-Expressing Cell Lines

The lentivirus-like particles expressing SARS-COV2 M-T2A-E, prepared in Example 1, were infected into a cell line capable of secreting exosomes by apoptosis, prepared according to Example 2, and a cell line expressing the SARS-COV2 M and E genes was prepared. To confirm the expression of the M and E genes, a cell line expressing SARS-COV2 M and E genes was prepared as a result of selecting and confirming 2 to 4 μg/ml of blastidin for 2 weeks from 48 hours after infection.

The cell line expressing the SARS-COV2 M and E genes was infected with lentivirus-like particles expressing the SARS-COV2-S 3R mutant, manufactured in Example 1, to prepare a cell line expressing the SARS-COV2 M, E, and S genes. In order to confirm the expression of M, E and S genes, 50 to 100 μg/ml of zeocin was selected for 2 weeks from 48 hours after the infection, and SARS-COV2 M, E and S genes were prepared.

In addition, the cell line expressing the SARS-COV2 M, E, and S genes was infected with lentivirus-like particles expressing SARS-COV2 N, manufactured in Example 1, to produce a cell line expressing the SARS-COV2 M, E, N, and S genes. In order to confirm the expression of M, E, N and S genes, 1 to 2 μg/ml of puromycin was selected for 2 weeks from 48 hours after the infection, and SARS-COV2 M, E, N and S genes were prepared.

### Example 4. Induction of Apoptotic Exosomes Containing SARS-COV-2 Structural Proteins

In order to apoptotic exosomes expressing SARS-COV-2 structural proteins, apoptosis was induced in the cell line prepared according to Example 3. Specifically, each cell line prepared according to Example 3 was cultured and then treated with 1 M staurosporine for 48 hours to induce apoptosis.

Afterwards, differential centrifugation was performed under the conditions described in Table 4 to separate the apoptotic exosomes from the cell culture supernatant. The apoptotic exosomes were then sonicated in PBS and finally obtained (FIG. 6).

TABLE 4

| Number | Differential centrifugation conditions |
|---|---|
| 1 | 300xg 10 min. |
| 2 | 2,000xg 20 min. Performed two times |
| 3 | 100,000xg 70 min, Performed two times |

### Example 5. Confirmation of the Expression of SARS-COV-2 Structural Proteins in Apoptotic Exosomes

In order to confirm the expression of SARS-COV-2 structural proteins in the apoptotic exosomes finally obtained in Example 4, exosomes were isolated from 293T cell lines expressing the SARS-COV-2 M, E, N, and S structural proteins and control 293T cell lines, and the expression of SARS-COV-2 structural protein and exosome marker CD63 from the same amount of protein was measured with a western blot.

Specifically, cell pellets and exosome pellets were suspended in lysis buffer (50 mM Tris-CI, pH 7.5, 150 mM NaCl, 1 mM EDTA, 1% Triton X-100, 1 mM Na3VO4, 1 mM NaF, 1 μg/mL pepstatin A, 10 μg/mL AEBSF, 2 μg/mL aprotinin, and 1 μg/mL leupeptin) and incubated on ice for 20 minutes. Then, centrifugation was performed for 20 minutes to obtain the protein suspension. The protein suspension solution was electro-transferred to PVDF-membrane after SDS-PAGE (sodium dodesyl sulfate-polyacrylamide gel electrophoresis). The PVDF membrane was overnight incubated at 4° C. with the primary antibody. At this time, the primary antibody was PA5-112048 (Invitrogen, 1:1000) for SARS-COV-2 S protein antibody, NBP 3-07059 (Novusbio, 1:1000) for SARS-COV-2 M protein antibody, NBP 3-07959 (Novusbio, 1:1000) for SARS-CoV-2 E protein antibody, MA5-36271 (Invitrogen, 1:1000) for SARS-COV-2 N protein antibody, and SC-5275 (Santa Cruz Biotechnology, 1:1000) for CD63 antibody. Thereafter, the secondary antibody peroxidase-conjugated secondary Ab (Pierce, 1:2, 500) was incubated at room temperature for 1 hour, and the protein band was detected by using enhanced chemiluminescence (ECL) detection.

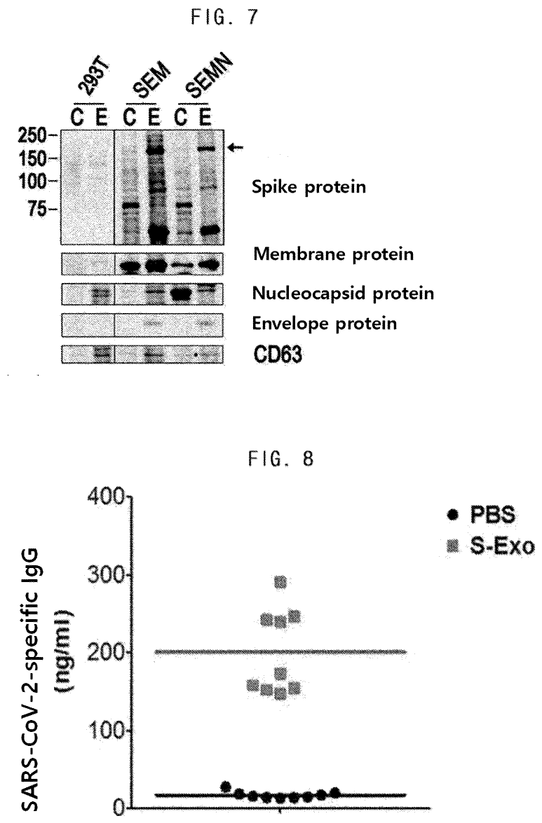

As a result, bands for the M, E, N, and S structural proteins were observed in the apoptotic exosomes (FIG. 7), confirming that apoptotic exosomes containing the SARS-COV-2 M, E, N, and S structural proteins were generated.

### Example 6. Confirmation of Superior Antibody Formation Capability of Apoptotic Exosomes Expressing SARS-COV2 Structural Proteins

Mouse experiments were performed to analyze the antibody formation ability of SARS-COV-2 structural proteins-expressing apoptotic exosomes prepared according to Example 4. Specifically, the apoptotic exosomes expressing SARS-COV-2 structural proteins were injected into the leg muscles of C57BL/6 mice at a dose of 10 μg to immunize them. Blood samples of about 300 to 400 μL were collected from the retro-orbital plexus and allowed to clot at room temperature for about 30 to 40 minutes and the serum from the clotted blood was collected and stored at temperatures below −20° C. Subsequently, the serum collected on the 14th day after immunization with exosomes was then used to analyze the levels of specific antibodies (IgG) against the S protein.

Specifically, in order to measure the levels of anti-SARS-COV2 IgG and isotype IgG (IgG1, IgG2a) in the serum, ELISA analysis was performed using an ELISA Kit (Abcam, ab284402). The S protein was used to coat the ELISA plate at 100 ng/well, followed by blocking the plate with a blocking solution containing BSA (bovine serum albumin). The standard area of the ELISA plate was coated with anti-mouse IgG-UNLB. The blocking ELISA plate's standard area was prepared by performing a 2-fold serial dilution starting with a known concentration of mouse IgG at 100 ng/ml in the first well. For the sample area, the prepared serum was added to the first well at a 200-fold dilution, followed by a 2-fold serial dilution across six steps. The ELISA plate with standard IgG and serum was incubated at 37° C. for 1 hour and then washed three times. An anti-mouse IgG antibody conjugated with HRP (horseradish peroxidase) was added to the washed ELISA plate and incubated for another hour, followed by another washing. Substrate buffer was then added to the ELISA plate to induce a color reaction. The color development was measured at 450 nm using an ELISA plate reader, and the amount of antibodies against the S protein in the serum was calculated by comparing it with the standard IgG.

As a result, antibodies were detected on the 14th day (FIG. 8) and it was confirmed that the mice had acquired immunogenicity, and it was confirmed that the antibody formation effect by SARS-COV-2 structural protein-expressing cytocytotropes exosomes was excellent.

The present invention has been described with a focus on its embodiments. Those skilled in the art will understand that the present invention can be implemented in modified forms without departing from its essential characteristics. Therefore, the disclosed embodiments should be considered in a descriptive rather than a restrictive sense. The scope of the present invention is indicated by the claims rather than the foregoing description, and all differences within the scope of the claims should be construed as included in the present invention.

1. An exosome comprising a viral structural protein.

2. The exosome of claim 1, wherein the exosome is a normal exosome and an apoptotic exosome.

3. The exosome of claim 2, wherein the apoptosis is induced by cleavage of a Gasdermin family protein.

4. The exosome of claim 3, wherein the cleavage is induced by staurosporine.

5. The exosome of claim 1, wherein the culture medium for obtaining the exosome comprises at least one substance selected from the group consisting of tumor necrosis factor alpha (TNF-a), Cycloheximide, Anisomycin, Aurintricarboxylic acid, Diphtheria toxin, Edeine, Fusidic acid, Pactamycin, Puromycin, Ricin, Sodium fluoride, Sparsomycin, Tetracycline, Trichoderma and staurosporine.

6. The exosome of claim 2, wherein the Gasdermin family protein is at least one protein selected from the group consisting of GSDMA, GSDMB, GSDMC, GSDMD, DFNA5 (GSDME) and DFNB59.

7. The exosome of claim 1, wherein the exosome is derived from a cell selected from the group consisting of stem cells, immune cells, somatic cells, fetal cells cell lines and tumor cells.

8. The exosome of claim 1, wherein the exosome is derived from a human cell line or an animal cell line.

9. The exosome of claim 1, wherein the structural protein is at least one protein selected from the group consisting of membrane, envelope, nucleocapsid and spike.

10. The exosome of claim 1, wherein the virus is SARS-COV-2.

11. A viral vaccine composition comprising the exosome of claim 1.

12. The viral vaccine composition of claim 11, wherein the virus is a coronavirus.

13. The viral vaccine composition of claim 12, wherein the coronavirus is SARS-COV-2.

14. A cell line for producing the exosome of claim 1.
